(19) United States
(12) Patent Application Publication
(10) Pub. No.: US 2009/0162447 A1
(43) Pub. Date: Jun. 25, 2009

(54) Spherically Shaped Substances

(76) Inventors: Tamar Kaully, Adi (IL); Moshit Yaskin-Harush, Mizpe Aviv (IL); Corinne Nadiv, Haifa (IL); Zachi Tenenbaum, Kiryat-Motzkin (IL); Yael Sides, Haifa (IL); Dganit Shacham, Nahariya (IL)

Correspondence Address: MARTIN D. MOYNIHAN d/b/a PRTSI, INC., P.O. BOX 16446, ARLINGTON, VA, 22215, US

(21) Appl. No.: 11/887,630

(22) PCT Filed: Mar. 30, 2006
(86) PCT No.: PCT/IL2006/000404

§ 371 Date: Nov. 17, 2008

Related U.S. Application Data
(60) Provisional application No. 60/708,766, filed on Aug. 17, 2005.
(60) Provisional application No. 60/708,765, filed on Aug. 17, 2005.

(30) Foreign Application Priority Data
Mar. 31, 2005 (IL) 167772

(51) Int. Cl. A61K 9/12; A61K 31/554; A61K 31/155; A61K 31/55; A23L 1/00

(52) U.S. Cl. 424/490; 514/211.07; 514/635; 514/217; 426/89

(57) ABSTRACT

A particle having a rounded shape and characterized by a substantially smooth surface is disclosed. The particle can be made of a food substance (e.g., nutritional substance or nutraceutical substance), a pharmaceutical (pharmaceutically active ingredient or pharmaceutically acceptable carrier) or a cosmetic substance.

### FIELD AND BACKGROUND OF THE INVENTION

The present invention relates to a particle having a rounded shape, and more particularly, a rounded shape particle made of a food (e.g., nutritional, nutraceutical), pharmaceutical or cosmetic substance and characterized by a substantially smooth surface.

Rounded shape particles are known in the art and are commonly used in the food and pharmaceutical industries in the preparations of nutritional, nutraceutical, pharmaceutical or other formulations.

With the current emphasis on health and fitness, there has arisen also an awareness of the need the human body has for proper nutrition. Particular emphasis has been placed on the need to balance diversified nutritional substances consumed by the individuals, and, if necessary to supplement the nutritional substances by nutraceutical substances such as vitamins and minerals.

Nutritional or nutraceutical substances are commercially available in many formulations including dry powders, liquids, emulsions, capsules, tablets and the like. When the formulation is prepared from dry powder, the quality of the formulation as well as its preparation efficiency, depends, not only on the production process and machinery, but also on the physical characteristics of the powder. This is due to the known processing difficulties associated with powder materials, including adherence of cohesive materials to containing surfaces, consolidation during transportation and storage and the like.

In order to utilize mass-production technology in the encapsulation of dry powder, for example, it is necessary that such compositions have desirable flow characteristics permitting rapid flow through high speed encapsulators without clumping or aggregation.

When dry powder formulations are used in the preparation of, e.g., nutritional, nutraceutical or pharmaceutical capsules or tablets, it is desired that the powder will exhibit enhanced packing characteristics to allow the use of automatic machinery.

The above characteristics are also desirable when the substances are marketed as powder or granular composition, as in the case of, for example, table sugar, cocoa, coffee, pediatric formulas, cosmetic products, pharmaceutical suspension powders and the like. For example, high flowability facilitates efficient preparation of beverages from the powder or granular composition.

In the pharmaceutical industry, the above characteristics are desirable in the preparations of suspensions or formulations containing excipients or carriers, e.g., preparations of controlled release products.

The advantages of controlled release preparations of therapeutic agents are well-established. When a drug release is non-controlled, the concentration of drug available in the bloodstream after administration quickly rises and then declines. Thus, it is of great advantage to both the patient and the physician that medication can be administered in a minimum number of daily doses from which the drug is released by a predetermined profile over a desired extended period of time.

This effect is accomplished using controlled release products. Controlled release products containing drugs, such as pharmaceutical medicaments or other active ingredients, are designed to contain higher concentrations of the drug and are prepared in such a manner as to effect controlled release of the drug into the gastrointestinal digestive tract of humans or animals over an extended period of time. Controlled release profiles include, for example, sustained release, prolonged release, pulsatile release and delayed release profiles. The use of controlled release products allows administration of fewer doses per day, makes patient compliance more likely and, for some controlled release profiles, reduces the frequency of swings of drug levels in the patient's system.

A controlled release product has to effect an effective dissolution of the drugs at the desired profile. Additionally, it is desired that such product will meet several other criteria, including uniformity of the product and simplicity as well as reproducibility of the manufacturing process.

Numerous techniques are known for preparing controlled release pharmaceutical forms. One technique involves surrounding an osmotically active drug core with a semipermeable membrane. The drug is released from the drug core over time by allowing body fluids (such as gastric or intestinal fluids) to permeate the membrane and dissolve the drug such that an efflux of the dissolved drug is generated.

Another common technique is to encapsulate a plurality of beads, pellets or tablets, coated with varying levels of a diffusion barrier or different types of the diffusion barriers.

In a process known as film coating, a uniform film is deposited onto the surface of a substrate. Because of the capability of depositing a variety of coating substances onto solid cores, this process has been used to make controlled release forms starting from different formulations, including tablets, granules, pellets, capsules and the like.

Many food powders and base or carrier substances in dosage formulations are provided in a form of spherical or spherical-like agglomerates of primary particles. The rounded shape of these agglomerates permits better packing, increasing the amount of material that can be packed in a given space. In the pharmaceutical industry, the rounded shape facilitates and enhances drug layering efficiency.

Also known are spherical or spherical-like non-agglomerated particles, such as microcrystalline cellulose spheres marketed by Asahi Kasei Chemicals, Japan (under trade name Celphere®) silica spheres, and sucrose-corn starch spheres, marketed by NP Pharm, France (under trade name Suglets®) and by Emilio Castelli, ITALY.

Sugar spheres are commonly used because they provide desirable functional properties. The low toxicity, high purity and diverse physicochemical properties of sugar account for its popularity in pharmaceutical applications.

Most spherical or spherical-like sugar particles, however, have a poor surface smoothness and oftentimes insufficient flowability. When, for example, such particles are subjected to drug-layering or being coated by release-controlling layer, it becomes impossible to control the thickness of the coating. This may result in large variation in the release rate of the final product.

Sugar spheres are traditionally produced by coating regular crystalline sucrose crystals with sugar syrup and a starch dusting powder. This process is lengthy, labor intensive and expensive. In particular it is difficult to tune the temperature and speed of the coating pan so as to prevent conglomeration. It has been reported that the tuning process may last up to 40-50 hours and must be performed by highly skilled and experienced personnel. Additionally, these sugar-starch spheres cannot be used in starch-free formulations.

Also of prior art of relevance are U.S. Pat. Nos. 5,376,386 and 6,780,508 and International Patent Application, Publication No. WO 03/094883. These publications independently disclose several crystalline sugars having rounded edges.

A known technique for treating particles for the purpose of reducing their size or shaping them is by ultrasound. Prior art techniques for treating particles by ultrasound were developed mainly in the explosive industry.

U.S. Pat. No. 5,035,363 to Somoza, the contents of which are hereby incorporated by reference, discloses a system for reducing the particle size of energetic explosive materials. Slurry containing the particulate explosive materials and an inert liquid, such as water or an aqueous solution, is subjected to intense acoustic cavitation from an ultrasonic generator for a short time. The particulate explosive materials are rapidly ground to a small particle size while minimizing the danger of detonation.

In U.S. Pat. No. 4,156,593 to Tarpley, Jr., the contents of which are hereby incorporated by reference, slurry of coal and a liquid including a leaching agent is directed through a chamber. The coal particles are comminuted and cavitation is induced in the slurry by contacting the slurry with a resonant vibration transmitting member. Thereafter, the liquid is separated from the comminuted particles.

In U.S. Pat. No. 6,669,122, the contents of which are hereby incorporated by reference, raw slurry of starting material is formed in a liquid which is a partial solvent of the material. The slurry is then exposed to treatment by ultrasound generators to produce ultrasound waves which shape and grind the starting particulate material. The particles are separated from their slurry by removing the partial solvent by decantation and/or filtration.

The above techniques, however, were not employed for food substance, pharmaceutical substance or other excipients or carriers. There is thus a widely recognized need for, and it would be highly advantageous to have a rounded particle, devoid of the above limitations.

### SUMMARY OF THE INVENTION

According to one aspect of the present invention there is provided a rounded particle, the particle is characterized by a substantially smooth surface.

According to another aspect of the present invention there is provided a composition which comprises a plurality of particles described herein.

According to yet another aspect of the present invention there is provided a formulation which comprises a pharmaceutical composition and the particle described herein.

According to still further features in the described preferred embodiments the particle is made of a substance which comprises a pharmaceutically active ingredient.

According to still further features in the described preferred embodiments the pharmaceutically active ingredient comprises diltiazem hydrochlorid. According to still further features in the described preferred embodiments the pharmaceutically active ingredient comprises metformin. According to still further features in the described preferred embodiments the pharmaceutically active ingredient comprises oxcarbazepine.

According to still further features in the described preferred embodiments the particle is coated by the pharmaceutical composition.

According to still further features in the described preferred embodiments the particle is mixed with the pharmaceutical composition.

According to still further features in the described preferred embodiments the pharmaceutical composition comprises at least one pharmaceutically active ingredient.

According to still further features in the described preferred embodiments the pharmaceutical composition comprises at least one pharmaceutically acceptable carrier.

According to still further features in the described preferred embodiments the particle is made of a substance which comprises at a pharmaceutically acceptable carrier.

According to still further features in the described preferred embodiments the particle is made of a substance which comprises a disaccharide. According to still further features in the described preferred embodiments the disaccharide is sucrose.

According to still further features in the described preferred embodiments the particle is made of a substance which comprises a monosaccharide. According to still further features in the described preferred embodiments the disaccharide is fructose.

According to still further features in the described preferred embodiments the particle is made of a substance which comprises a food substance.

According to still further features in the described preferred embodiments the particle is made of a substance which comprises a nutritional or nutraceutical substance.

According to still further features in the described preferred embodiments the particle is made of a substance which comprises a cosmetic substance.

According to still further features in the described preferred embodiments the particle is made of a substance which comprises cocoa.

According to still further features in the described preferred embodiments the particle is made of a substance which comprises an instant coffee component.

According to still further features in the described preferred embodiments the particle is made of a substance which comprises a vitamin.

According to still further features in the described preferred embodiments the particle is made of a substance which comprises a mineral.

According to still further features in the described preferred embodiments the particle is made of a substance which comprises a spice.

According to still further features in the described preferred embodiments the particle is made of a substance which comprises soy.

According to still further features in the described preferred embodiments the particle consists of a single substance.

According to still further features in the described preferred embodiments the particle is characterized by a non-layered filled structure.

According to still further features in the described preferred embodiments the rounded shape is an ellipsoid.

According to still further features in the described preferred embodiments the rounded shape is characterized by a sphericity of at least 80%, more preferably at least 85%.

According to still further features in the described preferred embodiments the particle has a specific surface area being lower than 0.1 m² per gram per 400 micrometer diameter.

According to still further features in the described preferred embodiments the substantially smooth surface is characterized by a roughness being lower than 1% of the diameter of the particle.

According to still further features in the described preferred embodiments a ratio between an optical surface area of the particle and a BET surface area of the particle is larger than 0.3.

According to still further features in the described preferred embodiments the composition is a dry powder composition.

According to still further features in the described preferred embodiments the dry powder composition is characterized by an angle of repose which is lower than 45 degrees.

According to still further features in the described preferred embodiments the dry powder composition is capable of maintaining a flow rate of at least 6 gram per square centimeter per second, through a 5 millimeters nozzle tilted at an angle of 30°.

The present invention successfully addresses the shortcomings of the presently known configurations by providing a particle of rounded shape which enjoy properties far exceeding the prior art.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-B are schematic illustrations of a prior art apparatus, for shaping slurry by ultrasound;

FIG. 2 is a schematic illustration of an apparatus for treating particles and liquids by ultrasonic cavitation, according to a preferred embodiment of the present invention;

FIG. 3 is a schematic illustration of a double walled vessel of the apparatus, according to a preferred embodiment of the present invention;

FIG. 4 is a schematic illustration of a liquid receiving container of the apparatus, according to a preferred embodiment of the present invention;

FIGS. 5A-B are schematic illustrations of a support framework, supporting ultrasound transducer elements of the apparatus, according to a preferred embodiment of the present invention;

FIG. 6 is a schematic illustration of a side view of a ultrasound transducer element secured to one of the walls of the liquid receiving container, according to various exemplary embodiments of the present invention;

FIGS. 7A-B are schematic illustrations of a perspective bottom view (FIG. 7A) and a side view (FIG. 7B) of the double walled vessel, according to various exemplary embodiments of the present invention

FIG. 8 is a schematic illustration of a prototype apparatus manufactured according to the teachings of preferred embodiments of the present invention and used for the production of various types rounded particles;

FIGS. 9A-C are images of: the raw sucrose particles (FIG. 9A), sucrose particles of the present embodiments (FIG. 9B) and the prior art particles (FIG. 9C);

FIGS. 10A-D are images of: 200-600 micrometer (FIGS. 10A-B), 600-1200 micrometer (FIG. 10C) and 100-250 micrometer (FIG. 10D) raw fructose particles;

FIG. 11A is an image of 200-600 micrometer rounded fructose particles prepared in a slurry of methanol and fructose, according to a preferred embodiment of the present invention;

FIG. 11B is an image of 200-600 micrometer rounded fructose particles prepared in a slurry of ethanol and fructose, according to a preferred embodiment of the present invention;

FIG. 11C is an image of 600-1200 micrometer rounded fructose particles prepared in a slurry of methanol and fructose, according to a preferred embodiment of the present invention;

FIG. 11D is an image of 600-1200 micrometer rounded fructose particles prepared in a slurry of ethanol and fructose, according to a preferred embodiment of the present invention;

FIG. 11E is an image of 100-250 micrometer rounded fructose particles prepared in a slurry of ethanol and fructose, according to a preferred embodiment of the present invention;

FIG. 12 is an image of raw diltiazem hydrochloride particles;

FIG. 13 is an image of rounded smooth diltiazem hydrochloride particles produced according to a preferred embodiment of the present invention;

FIG. 14 is an image of raw metformin particles;

FIG. 15 is an image of rounded smooth metformin particles produced according to a preferred embodiment of the present invention;

FIG. 16 is an image of raw oxcarbazepine particles;

FIG. 17 is an image of rounded smooth oxcarbazepine particles produced according to a preferred embodiment of the present invention;

FIGS. 18A-C are images of three samples of particles used as an input to image processing software;

FIG. 19 is an image showing raw sucrose particles after image processing; and

FIGS. 20A-C are histograms describing the sphericity for the particles of FIGS. 18A-C, respectively.

### DESCRIPTION OF THE PREFERRED EMBODIMENTS

The present embodiments comprise a particle having a rounded shape which can be made of a food substance (e.g., nutritional substance or nutraceutical substance), a pharmaceutical substance or a cosmetic substance. The particle of the present embodiments preferably has a substantially smooth surface hence enjoys superior physical properties. The particle of the present embodiments can serve as a pharmaceutically active ingredient or as a carrier for pharmaceutical formulations, including, without limitation, controlled released formulations, e.g., pellets. The particle of the present embodiments can also be used in the food industry for the preparation of nutritional or nutraceutical formulations, e.g., capsules, tablets, or powder or granular composition for beverages. The particle of the present embodiments can also serve as a carrier or base for cosmetic compositions, including, without limitation, a makeup composition. The present embodiments are further of a formulation incorporating the particle and an apparatus for shaping the particle.

The principles and operation of present embodiments may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

According to one aspect of the present invention there is provided a particle having a rounded shape. The particle can be made of any substance. Representative examples of suitable substances, include, without limitation, food (e.g., nutritional or nutraceutical) substances, pharmaceutical substances, cosmetic substances and the like. Representative examples of suitable substances, include, without limitation, disaccharide (e.g., sucrose, lactose), monosaccharide (e.g., fructose), vitamin (e.g., folic acid, ascorbic acid), mineral (e.g., calcium carbonate, magnesium hydroxide), spice (e.g., peppercorn, salt), cocoa, instant coffee component, soy, pharmaceutically active ingredients (e.g., Diltiazem Hydrochloride, Metformin, Oxcarbazepine), and various other substances including substances suitable for the preparation of cosmetic powders. The particle can be made of any combination of the above substances (for example, a cocoa component and sucrose component, a vitamin component and a mineral component), or it can consists of a single substance, without any supplements

The particle of the present embodiments has a rounded shape and is being characterized by a substantially smooth surface. The shape of the particle can be described by any of the known symmetric geometrical objects (e.g., an ellipsoid, a spheroid, a sphere), or a general shape which is not necessarily symmetric but is substantially devoid of sharp edges. Thus, for example, the particle can have a general shape for which different portions are described by different geometrical objects.

In various exemplary embodiments of the invention the particle is substantially non-agglomerated. That is, the particle of the present embodiments is a primary particle which is substantially devoid of smaller primary particles. When a plurality of particles of the present embodiments form a powder, the powder preferably contain less than 10% by weight, more preferably less than 5% by weight, more preferably less than 1% by weight agglomerates, most preferably substantially devoid of agglomerates.

As used herein, “an agglomerate” refers to coalesced lump of two or more primary particles adhered together in a three dimensional structure in which each particle is joined to at least one adjacent particle.

An agglomerate typically includes a particle binder material present therein as a discontinuous phase and is located in the form of bond posts linking adjacent particles.

Thus, according to a preferred embodiment of the present invention the particle is devoid of particle binder material.

Being formed of a plurality of particles, an agglomerate typically has certain porosity characteristics. Thus, according to a preferred embodiment of the present invention the particle has a porosity which is below 10%, more preferably below 5%, more preferably below 1% porosity, e.g., the particle is preferably not porous.

Before providing a further detailed description of the particle, as delineated hereinabove and in accordance with the present embodiments, attention will be given to the advantages and potential applications offered thereby.

Hence, it is recognized that the flowability of powder is sensitive to the shape and smoothness of the particles of the powder, with better flowability to particles having low roughness and minimal or no sharp edges. During the flow of particles with sharp edges or rough surface area in a conduit or container, the velocity vector of the individual particles is randomly changed due to inter-particle collisions, resulting in non-laminar flow and consequently jamming of particles in narrow passageways or sharp corners of the conduit or container. Conversely, the velocity field of flowing particles which are round and smooth is substantially laminar with minimal or no turbulences because variations in the velocity vector due to the inter-particle collisions are generally small.

It is further recognized that packing properties of round and smooth particles are better than other particles, because when the particles are round and smooth, the number of particles which can be packed within a given volume does not depend (or has a negligible dependence) on the orientation of the individual particles. On the other hand, when particles with sharp edges or rough surface are packed into a given volume, relatively large void volumes are formed between neighboring particles because the particles have random relative orientation, and the sharp edges or the roughness of their surface prevent them from further approaching one another.

Thus, the round shape and smoothness improves the flow and packing characteristics of the particles of the present embodiments, providing them with enhanced flowability and packability.

The particles of the present embodiments can thus be marketed as a powder or granular composition, e.g., for preparation of beverages or liquid pediatric formulas by dissolving a predetermined amount of the powder or granular composition in hot or cold liquid, such as water or milk. Thus, in this embodiment the particle is soluble. The advantage of the particles of the present embodiments for preparation of beverages is their enhanced flowability, preventing the particles to adhere to the surface of the container during the preparation.

The particle of the present embodiments can also be encapsulated in capsules or compressed into tablets as desired. The advantage of the nutraceutical particles of the present embodiments in such encapsulation or compression is their round shape and smoothness allowing better packing of the particles into the capsules or tablets. Additionally, the enhanced flowability of the particles makes the encapsulation or compression process more efficient, with minimal or no clumping or aggregation of particles in the encapsulation or compression machinery.

In any formulation, the particle of the present embodiments can be also as a food additive.

The phrase “food additive” as used herein includes any material intended to be added to a food product. The material can, for example, include an agent having a distinct taste and/or flavor or physiological effect (e.g., vitamins).

As used herein, the phrase “food product” describes a material consisting essentially of protein, carbohydrate and/or fat, which is used in the body of an organism to sustain growth, repair and vital processes and to furnish energy. Food products may also contain supplementary substances such as minerals, vitamins and condiments.

A food product containing the nutritional or nutraceutical particle of the present embodiments can also include additional additives such as, for example, antioxidants, sweeteners, flavorings, colors, preservatives, enzymes, nutritive additives such as vitamins and minerals, emulsifiers, pH control agents such as acidulants, hydrocolloids, antifoams and release agents, flour improving or strengthening agents, raising or leavening agents, gases and chelating agents, the utility and effects of which are well-known in the art.

Alternatively or additionally, the particle of the present embodiments can be used as an additive. For example, in one embodiment, the particle is a fructose particle.

As used herein “fructose particle” refers to a rounded and substantially smooth particle made of fructose, and optionally, but not obligatorily, one or more additional substances.

Fructose, also known as fruit sugar, is a ketohexose (C₆H₁₂O₆) monosaccharide which is considered the sweetest naturally occurring sugar. Fructose is present in notable quantities only in honey and a few fruits in its free monosaccharide form.

Fructose has many functional properties, such as sweetness, hygroscopicity, browning reactivity and preservability [Wolfgang Wach, Südzucker AG Mannheim/Ochsenfurt, Offstein: Fructose, in Ullmann's Encyclopedia of Industrial Chemistry, Edited by Wiley-VCH Verlag GmbH & Co. KGaA, Germany; Handbook of Sugars 2nd edition, edited by Harry M. Pancoast and W. Ray junk, Avi Publishing Company INC; 1980: 377-382, 411-412].

The functional properties of fructose make the fructose particle of the present embodiments a desirable food additive. The fructose particle of the present embodiments can thus be used as a flavor component or sweetener in dietary food, ice cream, beverages (soft drinks as well as alcoholic beverages), baked goods and the like.

Being the sweetest of the nutritive sweeteners, fructose is an ideal sweetening agent for dietary application. The sweetness level is reduced with increasing temperature, thus, fructose has a more effective application at normal or cool food temperatures. With respect to reactivity, fructose is very reactive with some amino groups and is the most reactive commercial sugar used in food products. Additionally, fructose is very soluble in aqueous solutions. This property is can be exploited for preparing high density syrups.

As a result of its exceptional sweetening power, the fructose particle of the present embodiments can be used as a sweetener in low-calorie food products. Fructose has synergetic effects with other sweeteners (such as sucrose), allowing a reduction in amount of sweetener and calories without a reduction in perceived sweetness.

The high solubility at low temperature and large freezing-point depression of fructose are particularly useful for ice cream and other frozen-dessert food products, because these properties influence product taste and texture. Fructose is superior to all nutritive sweeteners and humectants in controlling the water in frozen systems. The high solubility also makes the fructose particle of the present embodiments suitable for use in dry-mix beverages, or other formulations including, without limitation, sports drinks and reduced-calorie beverages. Fructose has the ability to mask bitter, metallic tastes and aftertastes, especially with intense sweeteners.

Fructose is also more soluble in alcohol than sucrose. Thus, the fructose particle of the present embodiments can be used in the production of sweetened alcoholic liqueurs with higher contents of dry substances.

With respect to baked food products, the fructose particle of the present embodiments is advantageous in its influence on moisture management which leads to extended shelf life, color development due to intense browning and flavor development.

Fructose has also synergistic effect with starches or other gel-forming products, making the fructose particle of the present embodiments useful for increasing gel strength and improved texture.

Fructose is a highly hygroscopic material. Its tendency to adsorb humidity causes flow difficulties in production facilities such as funnels, and severe agglomeration during storage. The advantage of the fructose particle of the present embodiments is that its round shape and substantially smooth surface significantly reduces the tendency of the fructose particle to adsorb water, hence improves the flow and storage properties thereof.

In various exemplary embodiments of the invention the particle is made of sucrose. As known, sucrose is a crystalline solid disaccharide composed of a glucose residue and a fructose, and can be utilized in pharmaceutical formulations. In the preferred embodiments in which the particle is made of a single substance, it can be made, e.g., exclusively of sucrose, without any additional substance. The particle of the present embodiments can also be made without a specific additional substance, e.g., starch which may be undesired for a particular usage. Thus, according to the presently preferred embodiment of the invention the particle is starch-free.

In various exemplary embodiments of the invention the particle is made of a pharmaceutically active ingredient.

A representative example of a pharmaceutically active ingredient suitable of the present embodiment is metformin. Metformin is a biguanide hypoglycaemic agent used in the treatment of non-insulin-dependent diabetes mellitus not responding to dietary modification. Metformin improves glycaemic control by improving insulin sensitivity and decreasing intestinal absorption of glucose. Metformin is a hygroscopic material, which is known to have reduced flow properties due to its tendency to adsorb humidity. The reduced flow properties of raw metformin particles cause difficulties during production of metformin formulations. For example, it is difficult to maintain rapid flow of metformin powder through funnels without clumping or aggregation. The advantage of the metformin particle of the present embodiments is that its round shape and substantially smooth surface significantly reduces the tendency of the metformin particle to adsorb water, hence improves the flow and storage properties thereof. Additionally, the metformin particle of the present embodiments has a moderate potency, allowing it to be pressed in high content to produce a tablet or capsule volume. The round shape and substantially smooth surface of the particle of the present embodiments enhances packing density, hence improves pressability and facilitates metformin tablet production.

Another representative example of a pharmaceutically active ingredient suitable of the present embodiment is oxcarbazepine. Oxcarbazepine is an anti-epileptics active ingredient which is a structural derivative of carbamazepine. Oxcarbazepine is regarded as the therapeutic drug of first choice for the treatment of convulsions and severe painful conditions. The commercially available dosage forms, such as tablets and syrups, are especially suitable for regularly recurring administration over a prolonged period of treatment in order to ensure a uniform concentration of active drug in the blood.

Due to the needle like shape of raw oxcarbazepine particles, they have relatively low flowability and pressability. Moreover, raw oxcarbazepine particles have less than optimal potency and so high quantity or concentration is needed to form a suitable tablet or capsule. The advantage of the oxcarbazepine particle of the present embodiments is that its round shape and substantially smooth surface significantly improves the flow properties, packing density and pressability thereof.

Another representative example of a pharmaceutically active ingredient suitable of the present embodiment is diltiazem hydrochloride. Diltiazem is a benzothiazine derivative possessing calcium antagonist activity. Diltiazem blocks the influx of calcium ions in smooth and cardiac muscle and thus exerts potent cardio-vascular effects. Diltiazem has been shown to be useful in alleviating symptoms of chronic heart disease, particularly angina pectoris and myocardial ischemia and hypertension, while displaying a low incidence of side effects. A variety of sustained release formulations containing diltiazem hydrochloride are commercially available. Similarly to raw oxcarbazepine particles, particles of diltiazem hydrochloride also posses relatively low flowability and pressability. The advantage of the diltiazem hydrochloride particle of the present embodiments is that its round shape and substantially smooth surface significantly improves the flow properties, packing density and pressability thereof.

Rounded particles are typically characterized quantitatively by a geometrical quantity known as sphericity, which generally quantifies the deviation of a particular geometrical shape from a perfect sphere.

Ideally, the sphericity of a three dimensional object is calculated by dividing the volume of the object to the volume of a sphere circumscribing the object. However, for some objects, the determination of the volume is difficult and oftentimes impossible. Therefore, for practical reasons, an alternative “two-dimensional” definition of sphericity is used. According to this alternative, the sphericity is defined as the ratio between the area of the projection of the object onto a certain reference plane and the area of a circle circumscribing the projection. For example, suppose that an image of the object is displayed on a planar display, then the planar display can be considered as a reference plane and the image of the object can be considered as the projection of the object on the reference plane.

Thus, denoting the area of the image by A and the perimeter of the image by P, the sphericity, s, can be defined as s=4πA/P². As will be appreciated by one of ordinary skill in the art, when the image is a perfect circle, A=π(P/2π)²=P²/4π and s=1. When the area of the image is 0 (i.e., the image is a line or a curve) s=0.

Unless otherwise defined, “sphericity,” as used herein, refers to two-dimensional sphericity.

It is recognized that the “two dimensional” sphericity is, to a good approximation, equivalent to the “three dimensional” sphericity (ratio of volumes), provided it is calculated and averaged over many particles (say 10 or more) or many different reference planes. In such event, starting from the “two dimensional” sphericity, s, the “three dimensional” sphericity can be defined as the cubic root of s².

According to a preferred embodiment of the present invention the sphericity of the particle is at least 80% more preferably at least 85%.

The particle of the present embodiments is preferably in the sub-millimeter size but can also be larger. A preferred diameter for the particle is from a few tens of micrometers to several thousands of micrometers, more preferably from about 100 micrometers to about 2 millimeters.

As used herein the term “about” refers to ±10%.

According to a preferred embodiment of the present invention the particle has a non-layered filled structure. As used herein, “non-layered filled structure” refers to a solid three dimensional structure whose cross section in any plane is devoid of any patterns in a form of successive layers of different materials and/or different texture.

In various exemplary embodiments of the invention the particle is characterized by a substantially smooth surface. Smooth surfaces can be characterized by low rugosity or conversely by high smoothness, which is commonly defined as the reciprocal to the rugosity of the surface.

The smoothness of the surface of the particle can be defined in more than one way.

In one embodiment, the smoothness is related to the surface area of the particle. Specifically for a given particle size, smaller surface area corresponds to higher smoothness. In this embodiment, the smoothness is conveniently defined in terms of specific surface area, defined as area per unit mass. As the specific surface area is a dimensional quantity, it should be supplemented by information regarding the size of the particle and the substance from which the particle is made.

Thus, according to the presently preferred embodiment of the invention, when the particle is made of sucrose, its specific surface area is lower than K m² per gram per 400 micrometer diameter, where K equals 0.2, more preferably 0.1, more preferably 0.08, more preferably 0.06, more preferably 0.04, even more preferably 0.03. As will be appreciated by one of ordinary skill in the art, the specific surface area depends on the average diameter of the particle. The above values of K are generally suitable for particles diameter of from about 300 to about 500 micrometers, which is conveniently averaged to 400 micrometers. For other particle diameters, the value of K should be rescaled using the average diameter of the particle, provided the average diameter has a sufficiently high accuracy (e.g., standard deviation of no more than 25%). Specifically, the value of K is rescaled using the square of the ratio between the respective average diameter and 400 micrometers. For example, for an average diameter D (expressed in micrometers), and standard deviation of no more than 25%, K should be replaced with K×(D/400)².

In another embodiment, the smoothness is defined by its contrast to the roughness of the surface. Specifically, low roughness corresponds to high smoothness. “Roughness” is a quantity which measures the irregularities of a surface, and is typically calculated using the distance, yᵢ, of the ith point on the surface (i=1, 2, . . . , N) from the mean surface y₀ which is the arithmetic average of all yᵢ's (y₀=(Σᵢyᵢ)/N). Two of the most commonly used definitions for roughness are the average roughness and the root-mean-square roughness. The average roughness is defined as the average of |y₀−yᵢ| over the perimeter of the particle. The root-mean-square roughness is defined as the square root of the average of |y₀−yᵢ|² over the perimeter of the particle. In any event, according to the presently preferred embodiment of the invention, the roughness (average roughness or root-mean-square roughness) of the particle is lower than 1% of the diameter of the particle, more preferably lower than 0.5% of the diameter of the particle, more preferably lower than 0.2% of the diameter of the particle.

In an additional embodiment, the smoothness is defined by a ratio between an optical surface area of the particle and a BET surface area thereof.

An optical surface area of the particle is the surface area of the particle as measured optically. Such measurement can be performed, for example, by obtaining an image of the particle using an optical microscope and estimating its surface area, by approximating the shape of the particle to a symmetrical shape. For example, when the shape of the particle is similar to a sphere, its optical surface area can be approximated by 4πR², where R is the radius of the particle, as measured from the image. When the shape of the particle is similar to a spheroid, having a long axis a and an aspect ratio of ε, its surface area can be approximated by 2πa² ε (ε+t/b), where b is given by b²=1−ε², and t (in radians) is given by sin(t)=b. As will be appreciated by one ordinarily skilled in the art the aspect ratio can also be approximated as the square root of the sphericity defined above.

A BET surface of the particle refers to a surface measured using a method developed by Brunauer, Emmett and Teller and commonly referred to as BET method. According to the BET method, the surface area is determined by allowing the surface to interact with nitrogen molecules and analyzing the corresponding adsorption curves. The BET method is well known in the art and is found in many publications (to this end see, e.g., S. J. Gregg and K. S. Sing, “Adsorption, Surface Area and Porosity”, Academic Press, London, 1995).

It was found by the Inventors of the present invention that the BET specific surface of a sucrose particle, manufactured according to the teaching of the present embodiment, can be about 0.03 gr/m² for particles whose average diameter is about 400 micrometer. The optical specific surface area of a spherical or a spherical-like sucrose particle of such diameter is 0.01-0.02 gr/m². In this numerical example, which is not to be considered as limiting, the ratio between the optical and BET surface area is from about 0.33 to about 0.66. In terms of rugosity, such range corresponds to a rugosity of 1.5-3. For comparison, the corresponding ratio for prior art sucrose-corn starch particles is from about 0.03 to about 0.07 (rugosity of 14.3-33.3), which is an order of magnitude lower than the sucrose particle of the present embodiment.

Thus, according to the presently preferred embodiment of the invention the ratio between the optical surface area and the BET surface area is larger than 0.3, more preferably larger than 0.4, most preferably larger than 0.6, say about 0.66, about 0.85, about 0.95 or more. In terms of rugosity, the particle of the present embodiment is preferably characterized by a rugosity which is below 3.33, more preferably below 2.5, most preferably below 1.67, say about 1.5, about 1.15, about 1.05 or less.

It is appreciated that when a plurality of particles of the present embodiments form a powder, the powder has an enhanced flowability. Powder flow behavior is multifaceted and complex. Yet, there is a variety of methods for characterizing the flowability of powder. Much research has been directed toward attempting to correlate the various measures of powder flow to manufacturing properties.

Generally, the flowability of powder can be characterized by a quantity known as “angle of repose”. One definition of the angle of repose is the constant, three-dimensional angle relative to a horizontal base that is assumed by a cone-like pile of material. To measure the angle of repose according to this definition, the powder of the present embodiments can be allowed to drop through a funnel onto a fixed, vibration-free base that includes a retaining lip to retain a layer of powder on the base. The height of the funnel is varied during the test in order to carefully build up a symmetrical cone of powder. Typically, the funnel height is maintained approximately 2 to 4 cm from the top of the powder pile as it is being formed in order to minimize the impact of falling powder on the tip of the cone. Alternatively, the funnel could be kept fixed while the base is permitted to vary as the pile forms. The angle of repose is determined by measuring the height of the powder cone and calculating the angle of repose. An angle of repose measured by this technique is oftentimes referred to as poured angle of repose.

Another definition of the angle of repose is the maximum angle of tilt of a bed of the powder beyond which the powder cannot retain a static pose. To measure the angle of repose according to this definition, the powder of the present embodiments can be placed on a smooth solid bed surface. The bed surface can then be tilted very slowly about the horizontal axis to a maximum angle without the particles sliding. When the bed surface is tilted beyond the maximum angle, sliding occurs, and the angle of the bed surface to the horizontal once the particles begin sliding is defined as the angle of repose. An angle of repose measured by this technique is oftentimes referred to as tilting angle of repose.

In various exemplary embodiments of the invention a plurality of particles of the present embodiments forms a powder characterized by an angle of repose which is below 45°, more preferably below 40°, say about 38°.

The flowability of the powder of the present embodiments can also be characterized by its flow rate at certain conditions. The flowability can be measured using a suitable flow meter, such as, but not limited to, a Hall flow meter. Thus, according to a preferred embodiment of the present invention the powder of the preset embodiments is capable of maintaining a flow rate of at least 6, more preferably at least 8, more preferably at least 10 gram per square centimeter per second, through a 5 millimeters nozzle tilted at an angle of 30°.

The particle of the present embodiments can be produced by subjecting raw material to ultrasound radiation, as described, for example, in U.S. Pat. No. 6,669,122.

Hence, the particle can be produced by forming slurry of raw substance, and treating the slurry by ultrasound radiation. The ultrasound radiation produces vibrations which shape the raw substance to produce rounded particles, characterized as further detailed hereinabove.

The generation of ultrasonic vibrations in the slurry results in cavitation and in the production of high local pressures. The high pressure in the cavities near the particles normally produces a grinding and shaping effects, reducing the particles' size and imparting to them a rounded shape. Whether the shaping or the grinding effect is predominant, depends on the frequency of the vibrations and energy density. Specifically, higher frequencies increase the shaping effect. Preferred ultrasound frequencies are from about 10 KHz to about 80 KHz, inclusive, more preferably from about 20 KHz to about 60 KHz, more preferably from about 25 KHz to about 50 KHz, inclusive. The particles can be separated from their slurry, e.g., by decantation and/or filtration.

It is generally preferred to stir the slurry during the process. Preferably, the stirring speed should be from 100 to 800 rpm. The ultrasound energy density is preferably from about 1 to about 50 Watts per liter.

As stated, the particle of the present embodiments can be used in a pharmaceutical formulation. In addition to the particle, the formulation can comprise any pharmaceutical composition, which can comprise one or more pharmaceutically active ingredient. Thus, the particle can be used as a carrier for the composition. The pharmaceutical composition can also comprise one or more pharmaceutically acceptable carriers in other forms. The formulation can be prepared either by coating the particle by the pharmaceutical composition, in which case the particle serves as a core or filler for the formulation. Alternatively, the formulation can be a prepared by addition the particle to the pharmaceutical composition as a filler so as to provide a mixture of the particle and the pharmaceutical composition.

When the particle of the present embodiments comprises a pharmaceutically active ingredient, it can include the pharmaceutically active ingredient per se or it can be coated by a layer of a pharmaceutically acceptable carrier. A formulation can be prepared by adding a pharmaceutically acceptable carrier to a powder formed of a plurality of particles of the present embodiments.

As used herein, a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Hereinafter, “pharmaceutically acceptable carrier,” refer to a carrier or a diluent that does not cause significant irritation to an organism and does not abrogate the biological activity and properties of the administered compound. An adjuvant is included under these phrases.

Herein, “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences,” Mack Publishing Co., Easton, Pa., which is herein fully incorporated by reference.

Pharmaceutical compositions of the present embodiments may be manufactured by processes well known in the art, e.g., by means of conventional mixing, dissolving, granulating, dragee-making, levigating, emulsifying, encapsulating, entrapping, or lyophilizing processes.

Pharmaceutical compositions for use in accordance with the present embodiments thus may be formulated in conventional manner using one or more pharmaceutically acceptable carriers comprising excipients and auxiliaries, which facilitate processing of the active ingredients into preparations that can be used pharmaceutically.

The pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

Pharmaceutical compositions suitable for use in the context of the present invention include compositions wherein the active ingredients are contained in an amount effective to achieve the intended purpose. More specifically, a “therapeutically effective amount” means an amount of active ingredients (e.g., a nucleic acid construct) effective to prevent, alleviate, or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the invention, the dosage or the therapeutically effective amount can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (See, e.g., Fingi, E. et al. (1975), “The Pharmacological Basis of Therapeutics,” Ch. 1, p.1.)

Dosage amount and administration intervals may be adjusted individually to provide sufficient plasma or brain levels of the active ingredient to induce or suppress the biological effect (i.e., minimally effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks, or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA-approved kit, which may contain one or more unit dosage forms containing the active ingredient. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of phammaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the invention formulated in a pharmaceutically acceptable carrier may also be prepared, placed in an appropriate container, and labeled for treatment of an indicated condition.

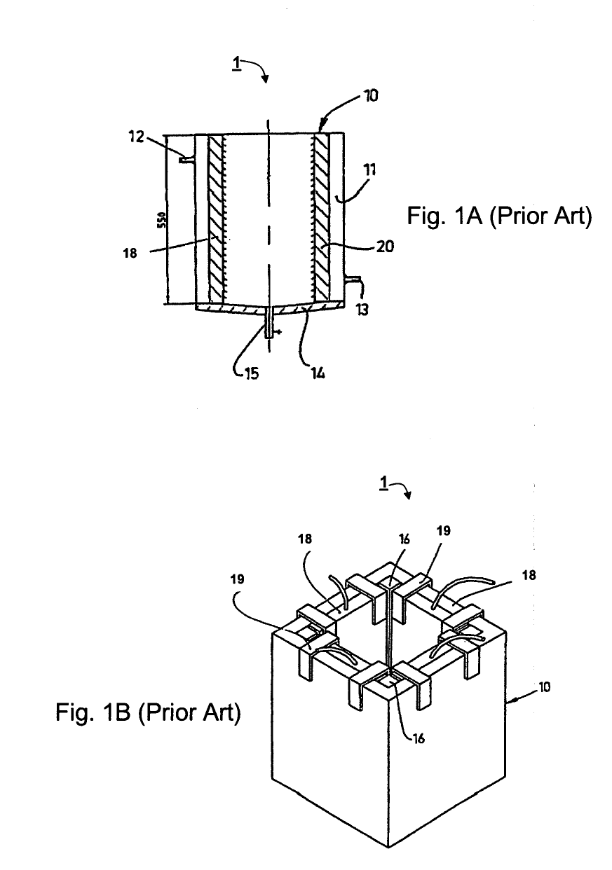

Referring now to the drawings, FIGS. 1A-B illustrates a prior art apparatus 1, for shaping slurry by ultrasound. Apparatus 1 includes a cavitation vessel 10 in which the cavitation and the shaping of the slurry occurs. Cavitation vessel 10 is provided with double wall 11, forming a space for a cooling fluid introduced through an inlet 12 and discharged through an outlet 13. Bottom 14 of vessel 10 is slanted to facilitate discharge of the slurry of shaped particles from the latter. An outlet 15 is provided in the center of slanted bottom 14. Numeral 16 designates posts which occupy spaces that are not in the effective zone for the cavitation process. Ultrasound transducers 18 are mounted by means of hooks 19 on cavitation vessel 10. The ultrasound generators may be of any type, such as known in the art. The ultrasonic vibrations gradually transform the raw slurry to a shaped slurry.

A major limitation of prior art apparatus 1 is that the anterior of vessel 10 is not adapted to allow uniform distribution of ultrasound energy therein. Particles present in the slurry oftentimes reach regions in which the energy density is rather low, and are therefore not affected by the cavitation. In particular, the existence of “dead zones” (posts 16) presents a drawback, whereby particles may accumulate therein and not participate in the process. Additionally, particles also tend to accumulate below transducers 18 which are only mounted on vessel 10 from above. Even when transducers 18 are positioned closely to one another and to the base of the vessel, they do not completely block the flow of slurry into posts 16 or below transducers 18. Moreover, as the transducers are mounted by hooks to the upper side of vessel 10, the dynamic process of ultrasonic cavitation together with rapid stirring, generates vibrations which in turn dislocate the transducers. As a result of the dislocation, gaps are formed between the transducers, and more slurry is accumulated in the “dead zones”.

While conceiving the present invention it has been hypothesized and while reducing the present invention to practice it has been realized that the particle of the present embodiments can be manufactured by an apparatus which is an improvement of apparatus 1.

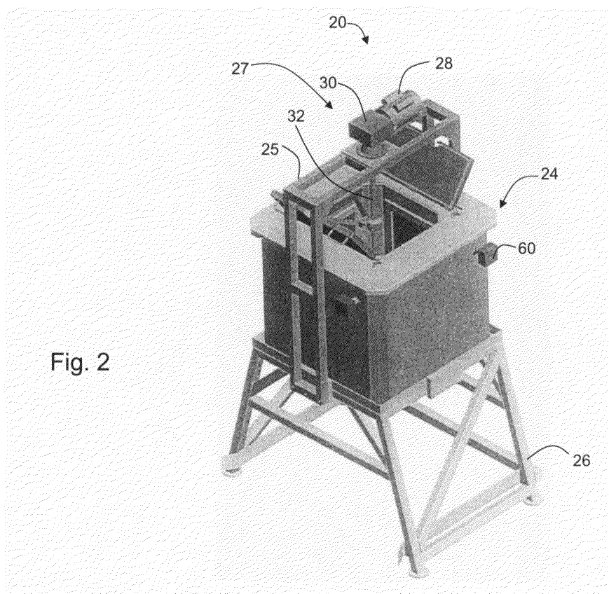

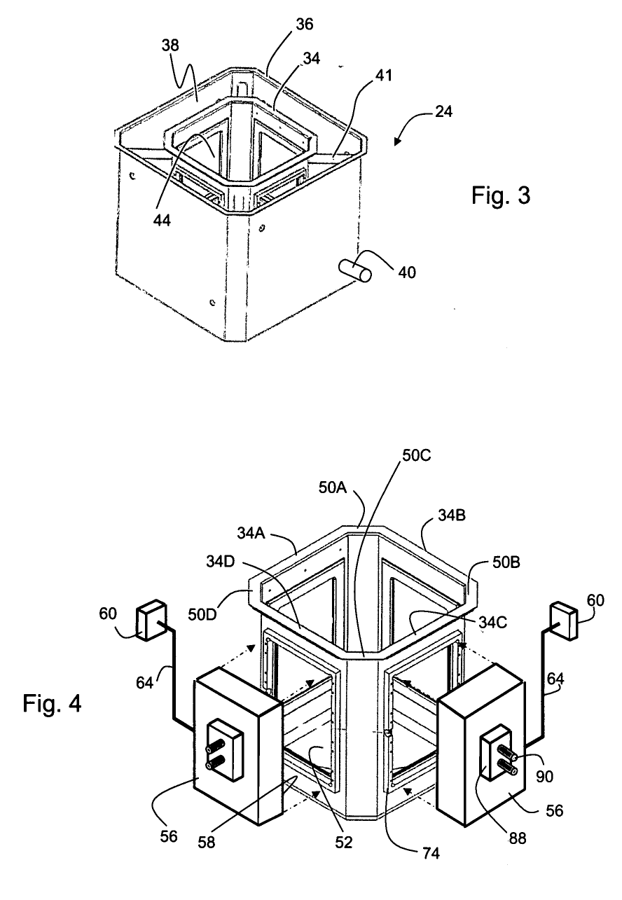

FIG. 2 illustrates an overall view of an apparatus 20 for treating particles and liquids by ultrasonic cavitation, according to a preferred embodiment of the present invention. Apparatus 20 preferably comprises a double walled vessel 24 which is optionally mounted on a support structure 26. Vessel 24 is better illustrated in FIG. 3. According to a preferred embodiment of the present invention vessel 24 comprises a liquid receiving container 34 surrounded by an exterior container 36 with a chilling liquid conduit 38 extending therebetween. Conduit 38 is preferably liquid tight and has a temperature reducing arrangement, e.g., in the form of a coil extending within conduit 38 for heat exchange via a cooling liquid flowing through the coil (not seen but only an inlet segment thereof 40 in FIG. 2). Extending between containers 34 and 36 are a plurality of supporting and reinforcing ribs 41. Preferably, receiving container 34 is substantially devoid of any laterally extending surfaces, such that when slurry or liquid is applied therein, a uniform dispersion of particles present in the slurry or liquid is maintained. Containers 34 and 36 preferably coaxially extend within one another such that container 34 gives rise to a treating zone 44.

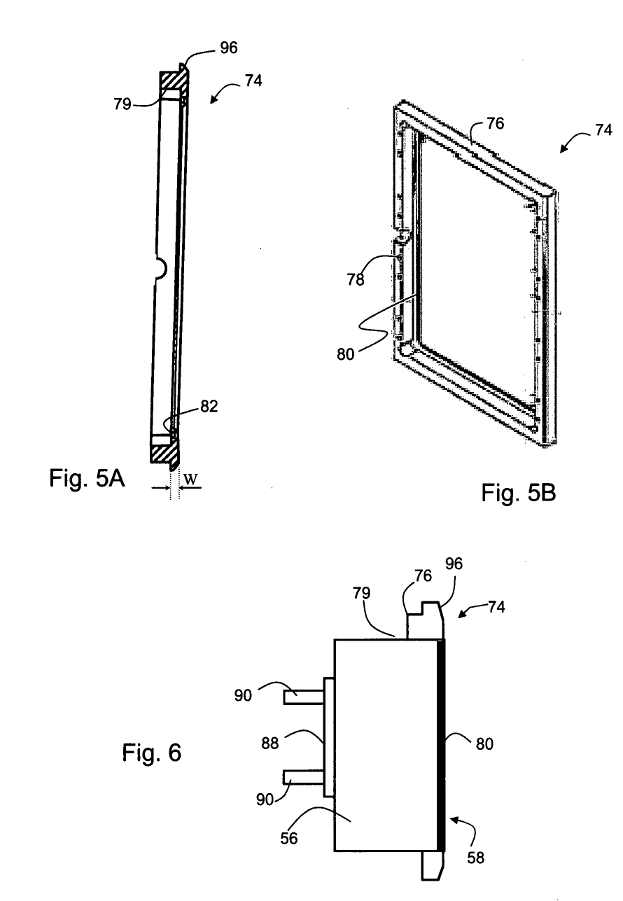

Container 34 is better illustrated in FIG. 4. In the preferred embodiment illustrated in FIG. 4, container 34 has four side walls, designated 34A, 34B, 34C and 34D, interconnected to one another with chamfered wall portions 50A-50D. However, this need not necessarily be the case as for some applications it may be desired to manufacture container 34 with a different number of side walls and/or different number of chamfered wall portions. The inner surfaces of the side walls and interconnecting chamfered wall portions of container 34 are substantially smooth. Additionally, the walls and the chamfered wall portions are preferably made of an acoustically reflective material. Preferably, the material is also selected so as not to interact with the liquid in the container. A representative example of a material suitable for the walls of the container is stainless steel metal or like material.

According to a preferred embodiment of the present invention at least one of the number, shape and orientation of the walls and chamfered wall portions is designed and constructed such that the acoustic reflections therefrom result in a substantially uniform distribution of acoustic field within the container. This embodiment is particularly advantageous because it prevents the formation of “dead zones” at the corners of the container.

Formed in each side wall there is a window 52 which accommodates an ultrasonic transducer element 56. Two transducer elements are shown in the schematic illustration of FIG. 4, but this should not be considered as limiting as the ordinary skilled person would know how to use the illustration to construct an apparatus having more transducer elements. Windows can also be formed in one or more of the chamfered wall portions, if desired. Transducer elements 56 are each associated with an ultrasound generator 60 extending outside of vessel 24 by means of conduit 64.

According to a preferred embodiment of the present invention, apparatus 20 comprises a stirring mechanism, generally shown by 27, for establishing a motion, typically rotary motion, of the slurry or liquid so as to ensure homogeneous dispersion of the particles within container 34 and uniform exposure of said particles to ultrasonic energy. The stirring mechanism is shown in FIG. 2 in a form of a bridge 25 supporting a motor 28 coupled to a gear unit 30 from which extends downwardly an axle 32 fitted with one or more steering blades (not shown). Axle 32 is preferably detachable so as to allow the replacement of the steering blades and/or for maintenance. Alternatively or additionally, the stirring mechanism can provide a stream of gas which is circulated in the slurry or liquid.

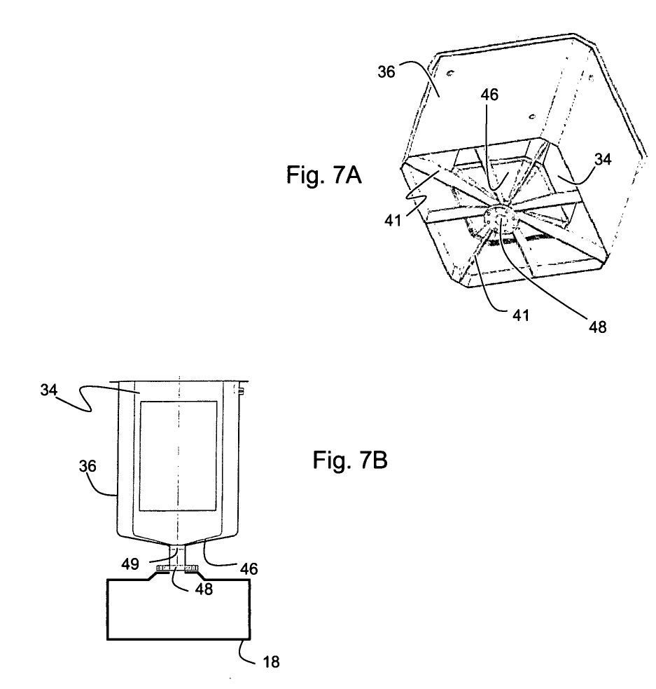

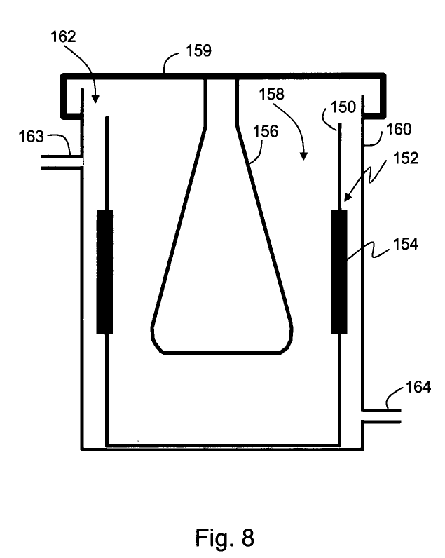

Elements 56 are preferably supported by means of a support framework 74, secured to the respective windows of container 34. Support framework 74, is better illustrated in FIGS. 5A-B (see also FIG. 6). In various exemplary embodiments of the invention support framework 74, comprises an external support peripheral rim 76 fitted for comfortably receiving within the window and secured in place by means of bolts 78. An inner peripheral rim 79 is fitted for receiving transducer element 56 which tightly bears against a sealing gasket 80 received within a suitable peripheral groove 82. Transducer element 56 is preferably secured to framework 74 by means of a bracing plate 88 tightened to framework 74 by bolts 90. The arrangement is such that fastening bolts 90 increases tight bearing of the front surface 58 of transducer element 56 against gasket 80 to thereby ensure a liquid tight engagement therebetween. In accordance with a different embodiment (not shown), the assembly comprises the support framework and the associated transducer elements 56 are tightly secured in place by means of a support flange secured directly to the outer surface of the side wall of the receiving container by means of suitable tightening bolts.

The periphery of framework 74 preferably has a slanted inner edge 96 (chamfered in the schematic illustrations of FIGS. 5A and 6 but it may just as well be rounded). The width, W, of the portion projecting inward from the inside surface of side walls 34A-34D is preferably of minimal dimensions so as to eliminate or reduce the problem of particles of material accumulating thereon during the process.

In the schematic illustrations of FIGS. 4 and 6, transducer elements 56 have a flat front surface 58. However, this need not necessarily be the case, since, for some applications, it may be desired for the transducer elements to have curve front surfaces. In such cases, the side walls of container 34 are preferably also curved.

In any event, the front surfaces of the transducer elements are preferably flush with the inner surfaces of the side walls, such that inner surface of the container is substantially smooth. Thus, according to a preferred embodiment of the present invention the cross section of container 34 is shape-wise compatible with the shape of elements 56. Specifically, when front surfaces 58 of elements 56 are flat, the inner surface of the walls of container 34 are also flat, whereby front surface 58 is substantially parallel, more preferably coplanar with the inner wall of container 34. Alternatively, when front surfaces 58 are curved, the containers have a generally round cross section (e.g., cylindrical containers), such that the curvatures of front surface 58 substantially match the curvature of the inner wall of container 34. In this embodiment, front surfaces 58 are substantially co-surfaced with the inner wall of container 34. In other words, elements 58 are positioned such that there is a minimal or no protrusion of front surface 58 inwards into container 34.

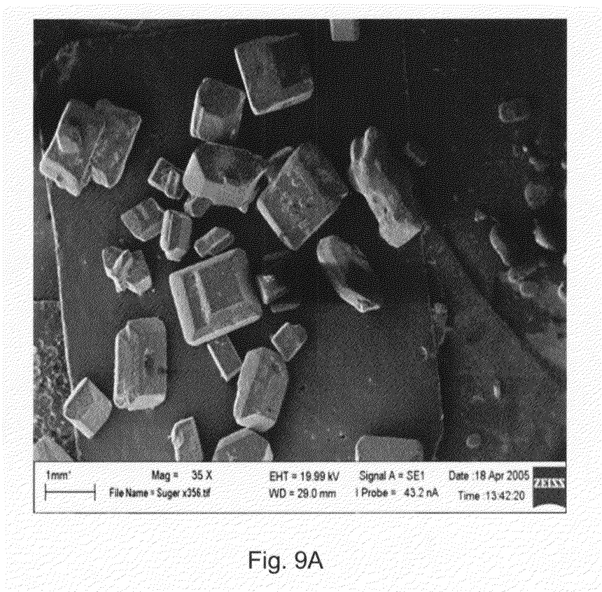

FIGS. 7A and 7B schematically illustrate a perspective bottom view (FIG. 7A) and a side view (FIG. 7B) of containers 36 and 34, according to various exemplary embodiments of the present invention. The base 46 of container 34 preferably has downwardly inclined surfaces extending towards a draining port 48 which, at the assembled position of apparatus 20, can extends above a collecting container 18. A spigot 49 preferably accommodates port 48 so as to allow control over liquid flow out of container 34. In use, the slurry or liquid to be treated can be poured directly into treating zone 44 of liquid receiving container 34, while spigot 49 is in a closed state preventing the slurry or liquid from exiting container 34. In this embodiment, once the ultrasound treatment is completed, spigot 49 is brought to an open state and the treated slurry or liquid is allowed to flow into collecting container 18.

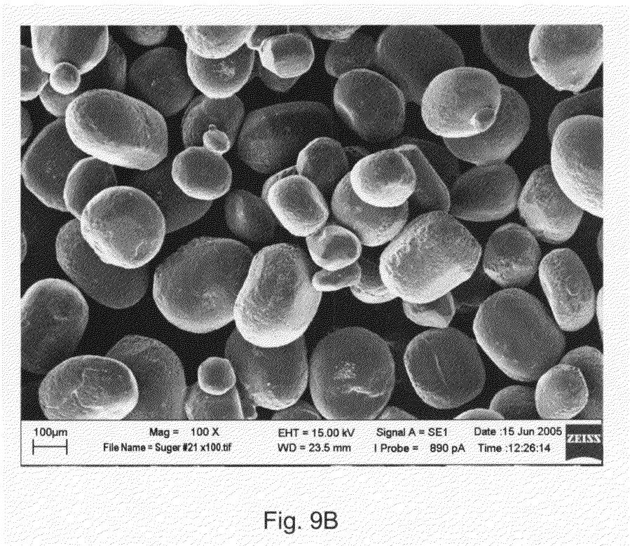

In alternative embodiment, an additional container, such as, but not limited to, an erlenrmeyer flask (not shown, see FIG. 8) can be secured to base 46 or rim 76, and the slurry or liquid is poured into the additional container. In this embodiment, the volume between the additional container and the walls of container 34 is preferably filled with a liquid medium (e.g., water), mediating transducer elements 56 and the slurry or liquid to be treated. The mediating liquid medium facilitates propagation of the acoustical field from transducer elements 56 to the slurry or liquid.

The ultrasound treatment of the slurry or liquid is preferably performed by activating the ultrasound generators to produce ultrasonic vibrations in the slurry or liquid. At the same time, the stirring mechanism is preferably activated to generate rotary motion to the particles in the slurry while being subjected to the acoustic field. Preferably, but not obligatorily, the stirring speed should be from about 100 to about 800 rpm.

The generation of ultrasonic vibration field in the slurry or liquid results in cavitation and in the production of high local pressures. The high pressure in the cavities, near the particles in the slurry, produces various effects including grinding or shaping of particles, enhancement of chemical reactions, ionization, erosion and the like.

Additional objects, advantages and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions illustrate the invention in a non limiting fashion.

### Example 1

### Production of Rounded Sucrose Particles

A prototype apparatus was built according to the teaching of the present embodiments and used for producing rounded particles of various substances. In the present Example, the prototype apparatus was tested for the production of rounded sucrose particles.

FIG. 8 illustrates the experimental system, which included a liquid receiving container 150, made of stainless steel and formed with windows 152 on its side walls. The windows accommodated flat ultrasonic transducers 154 extending coplanar with the surfaces of the side walls.

Liquid receiving container 150, was surrounded by an exterior container 160 so as to form a conduit 162 between the walls of the inner and exterior containers. Through an inlet 163, conduit 162 was filled with a chilling liquid which was continuously circulated via outlet 164 so as to maintain a constant temperature of about 30° C. within container 150.

150 liter of 90% methanol and 10% water were poured into container 150, and 50 Kg. of sucrose were added to the liquid in the container thus forming sucrose slurry by stirring.

The ultrasonic transducers were activated while stirring the slurry at a frequency of 25 kHz and a power of 2.5 kW, thus generating a power density of 0.015 kW/liter within the container. Following about 8 hours of treatment, the slurry was filtered under vacuum, and the sucrose was washed in methanol and dried at room temperature. A powder consisting of rounded smooth sucrose particles having average size of 300-600 micrometer was formed.

Following drying, a sample of the formed sucrose particles was subjected to BET measurement using a TriStar 3000 analyzer (Micromeritics, Georgia, USA), to obtain a specific surface area for the particles (surface area per unit mass). An averaged value of 0.03 m²/gr was measured. For comparison, the specific surface area of a sample of prior art particles of similar size (355-500 micrometer Suglets®, purchased from NP Pharm, France), was also measured. The specific surface area of the particles of this sample was 0.27 m²/gr. Thus, the sucrose particles produced according to the teaching of the present embodiments of the invention are characterized by a specific surface area which is about 9 times smaller than the specific surface area characterizing the prior art. As the average size of the samples was similar, the specific surface was used as a measure of the smoothness of the particles. Thus, it was demonstrated that the particles produced according to the teaching of the present embodiment are 9 times smoother than the prior art particles.

An additional estimate of the particles' smoothness was performed by analyzing the samples via Scanning Electron Microscopy (SEM).

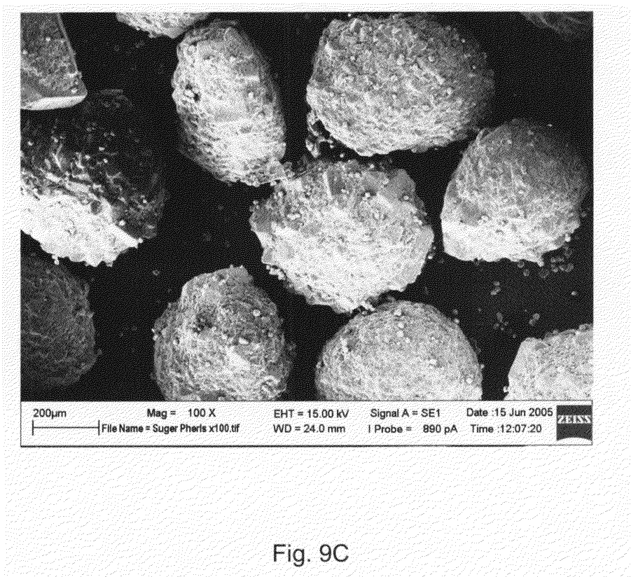

FIGS. 9A-C are images of the raw sucrose particles (FIG. 9A), sucrose particles of the present embodiments (FIG. 9B) and the prior art particles (FIG. 9C). Whereas the prior art particles revealed an average surface roughness of several micrometer, the surface of the particles prepared according to the teaching of the present embodiments was substantially smooth.

### Example 2

### Production Rounded Fructose Particles

Rounded fructose particles were prepared in two experiments, using an experimental system similar to the system shown in FIG. 8 and described in Example 1 hereinabove. In this example, erlenmeyer flask 156 was fastened to a external support peripheral rim 159 mounted above container 150.

In the first experiment, a 500 ml erlenmeyer flask was used. 200 ml of methanol were poured into the erlenmeyer flask, and the volume 158 between the erlenmeyer flask and the inner walls of container 150 was filled with water. 70 gr. of fructose were added to the liquid in the erlenmeyer flask, thus forming fructose slurry. Two sizes of raw fructose particles were used: 200-600 micrometer, and 600-1200 micrometer.

The ultrasonic transducers were activated while stirring the slurry. It was found by the inventors of the present invention that frequencies from about 16 kHz to about 60 kHz, and power densities from about 0.005 to about 0.05 kW/liter are suitable. Yet, higher efficiency was achieved at a frequency of about 25 kHz and a power density of about 0.01 kW/liter. During the process, a constant temperature of about 30° C. was maintained in liquid receiving container.

Following about 3 hours of treatment, the slurry was filtered under vacuum, and the fructose was washed in methanol and dried at room temperature. A powder consisting of rounded smooth fructose particles was formed.

In the third experiment, a 3 liter erlenmeyer flask was fastened to the rim of the liquid receiving container. 1.5 liters of ethanol were poured into the erlenmeyer flask, and the volume between the erlenmeyer flask and the inner walls of the liquid receiving container was filled with water. 500 gr of fructose were added to the liquid in the erlenmeyer flask, thus forming fructose slurry. Three sizes of raw fructose particles were used in the first experiment: 100-250 micrometers, 200-600 micrometers and 600-1200 micrometers.

The ultrasonic transducers were activated while stirring the slurry, as in the first and second experiments. Following about 3 hours of treatment, the slurry was so filtered by centrifuge and dried by rotary drum supplied with dry air. Powders consisting of rounded smooth fructose particles were formed.

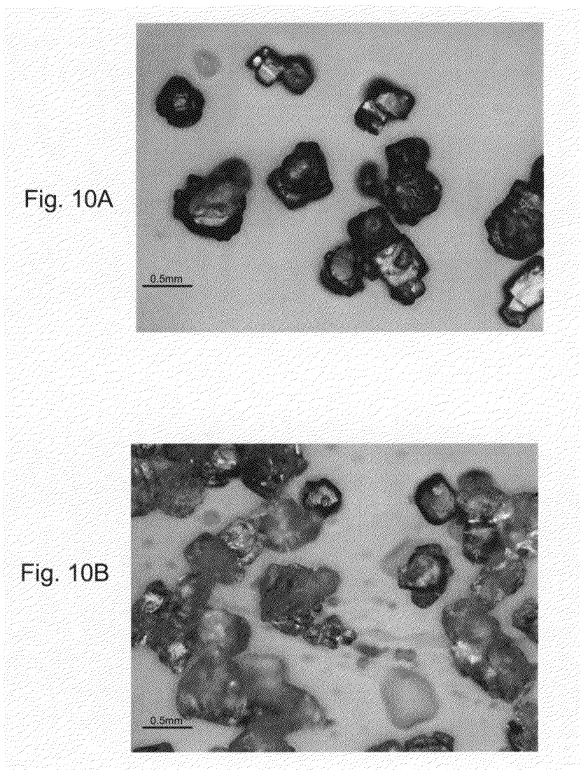

FIGS. 10A-D are images of the 200-600 (FIGS. 10A-B), 600-1200 (FIG. 10C) and 100-250 (FIG. 10D) micrometer raw fructose particles.

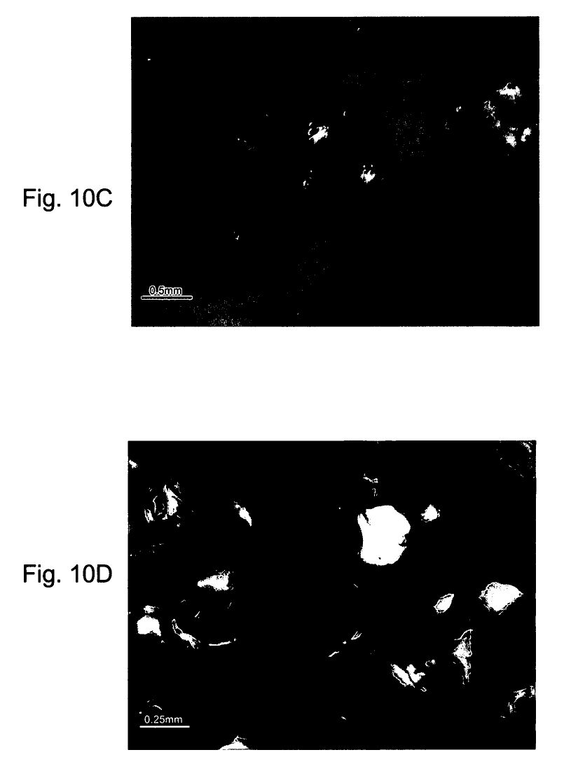

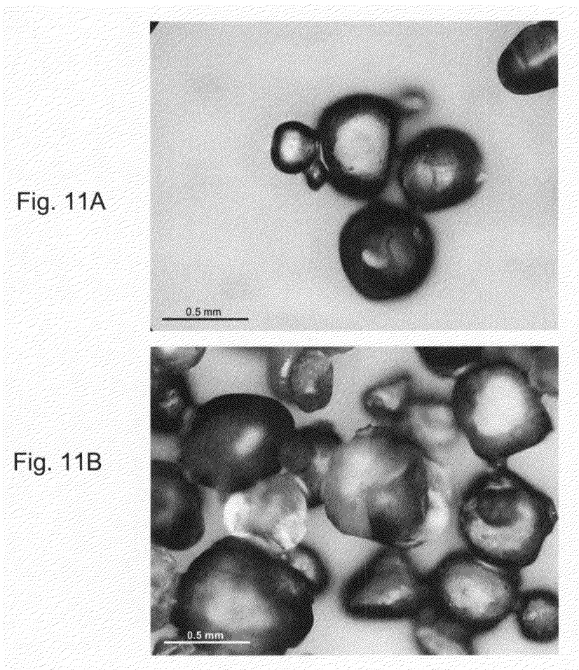

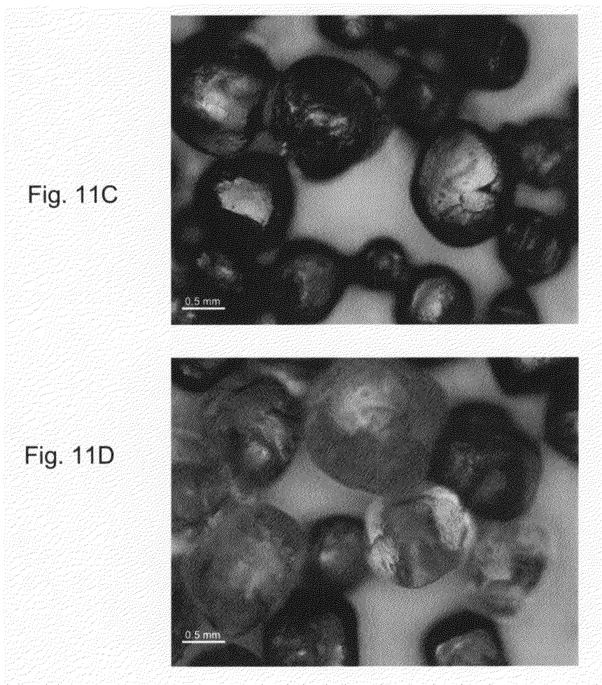

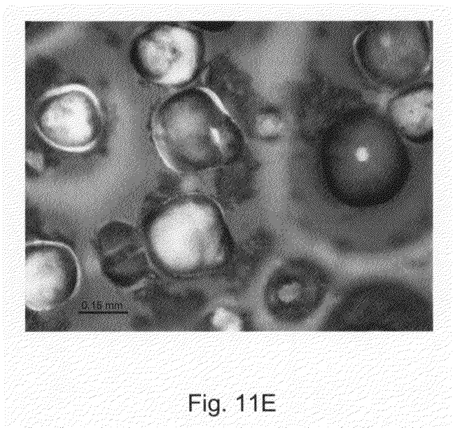

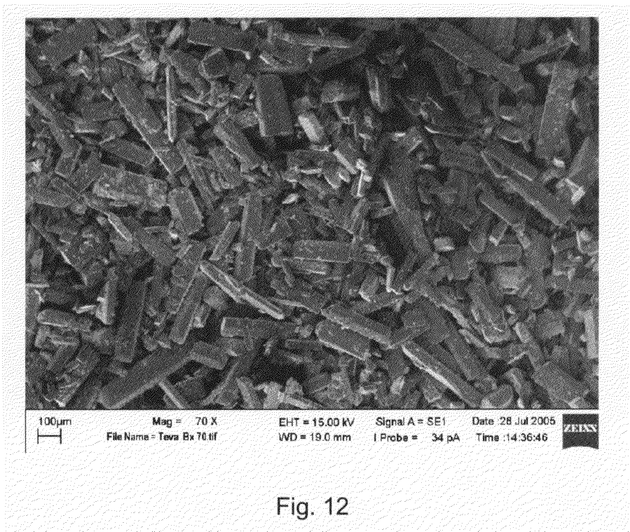

The rounded smooth fructose particles are shown in FIGS. 11A-E.

FIGS. 11A-B are images of 200-600 micrometer rounded fructose particles of the methanol experiment (FIG. 11A), and the ethanol experiment (FIG. 11B).

FIGS. 11C-D are images of 600-1200 micrometer rounded fructose particles of the methanol experiment (FIG. 11C), and the ethanol experiment (FIG. 11D).

FIG. 11E is an image of 100-250 micrometer rounded fructose particles of the ethanol experiment.

### Example 3

### Production of Rounded Diltiazem Hydrochloride Particles

Rounded diltiazem hydrochloride particles were prepared using an experimental system similar to the system shown in FIG. 8 and described in Examples 1 and 2 hereinabove.

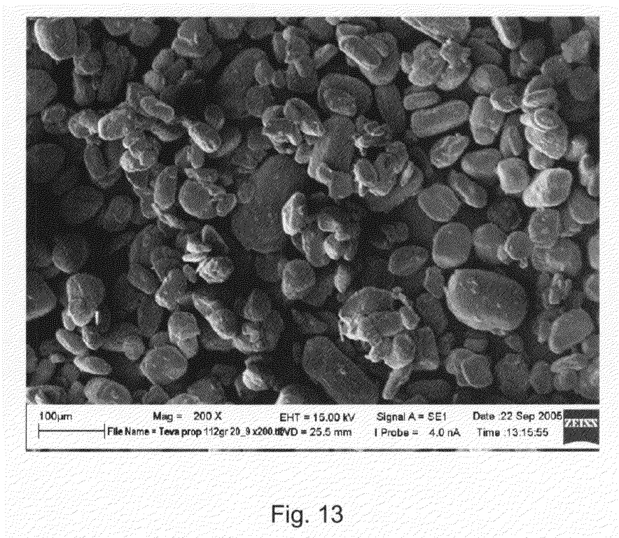

A 3 liter erlenmeyer flask was fastened to the rim of the liquid receiving container. 1.5 liter of propanol was poured into the erlenmeyer flask, and the volume between the erlenmeyer flask and the inner walls of the liquid receiving container was filled with water. 400 gr. of raw diltiazem hydrochloride particles were added to the liquid in the erlenmeyer flask to form slurry. The size of the raw particles was 20-45 micrometer. FIG. 12 is an image of the raw diltiazem hydrochloride particles.

The ultrasonic transducers were activated while stirring the slurry. It was found by the inventors of the present invention that frequencies from about 20 kHz to about 60 kHz, and power densities from about 0.005 to about 0.05 kW/liter are suitable. Yet, higher efficiency was achieved at a frequency of about 47.5 kHz and a power density of about 0.01 kW/liter. During the process, a constant temperature of about 30° C. was maintained in liquid receiving container.

Following about 2.5 hours of treatment, the slurry was filtered through a 20 micrometer sieve and a 45 micrometer sieve, and the particles were dried by air. A powder consisting of rounded smooth dilitiazem hydrochloride particles was formed.

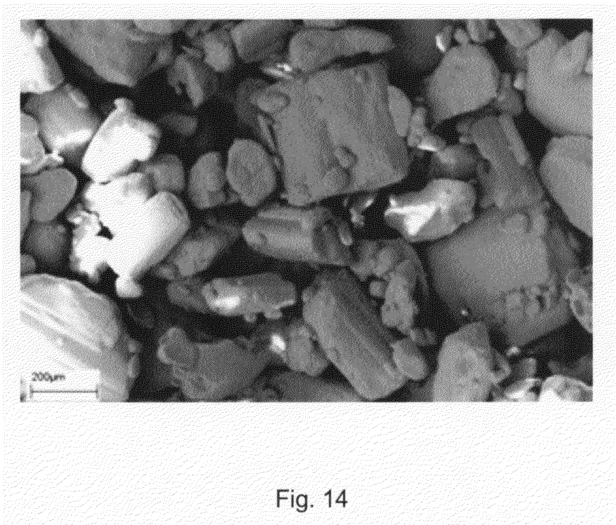

FIG. 13 is an image of 45 micrometers rounded smooth diltiazem hydrochloride particles produced in the experiment. As shown, the surface of the particles prepared according to the teaching of the present embodiments was substantially smooth.

The diltiazem hydrochloride particles were subjected to an angle of repose test. The particles were placed on a smooth tile positioned horizontally. The tile was tilted slowly each time at 1° about the horizontal axis. The angle of repose was defined as the angle at which the first particles had slide. The measured angle of repose was 48° for the powder containing the raw particles, and 38° for the powder containing the rounded smooth diltiazem hydrochloride particles, demonstrating a significant improvement of flowability in the rounded particles, compared to the raw particles.

### Example 4

### Production of Rounded Metformin Particles

Rounded metformin particles were prepared using an experimental system similar to the system shown in FIG. 8 and described in Examples 1 and 2 hereinabove.

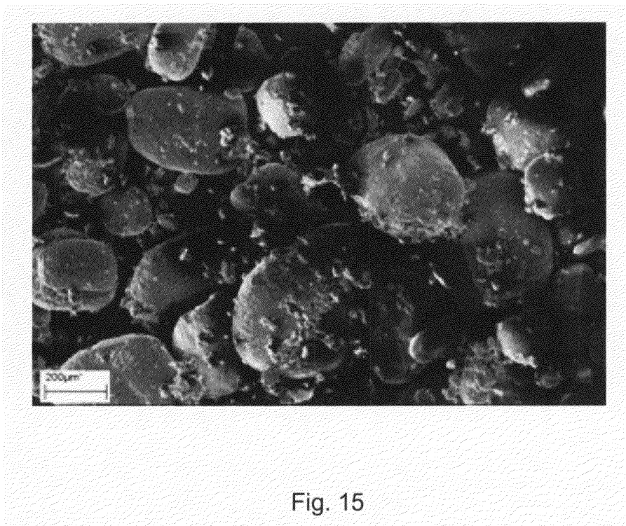

A 3 liter erlenmeyer flask was fastened to the rim of the liquid receiving container. 2.5 liter of methanol was poured into the erlenmeyer flask, and the volume between the erlenmeyer flask and the inner walls of the liquid receiving container was filled with water. 700 gr. of raw metformin particles were added to the liquid in the erlenmeyer flask form slurry. The size of the raw particles was 300-600 micrometer. FIG. 14 is an image of the raw metformin particles.

The ultrasonic transducers were activated while stirring the slurry. It was found by the inventors of the present invention that frequencies from about 16 kHz to about 60 kHz, and power densities from about 0.005 to about 0.05 kW/liter are suitable. Yet, higher efficiency was achieved at a frequency of about 25 kHz and a power density of about 0.01 kW/liter. During the process, a constant temperature of about 30° C. was maintained in liquid receiving container.

Following about 2 hours of treatment, the slurry was filtered under vacuum and the powder was dried at room temperature. A powder consisting of rounded smooth metformin particles was formed.

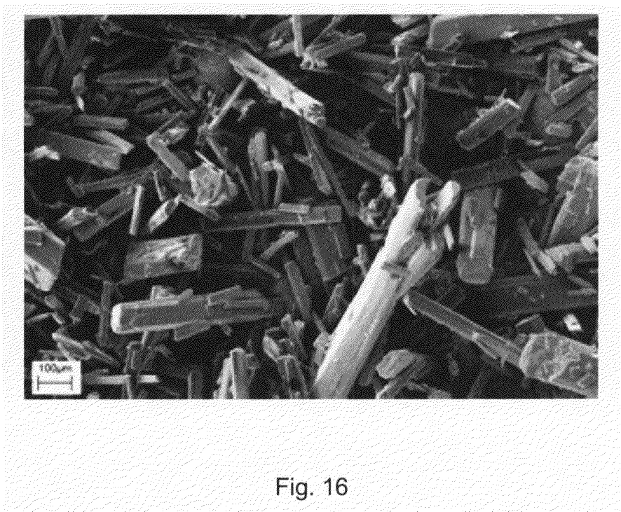

FIG. 15 is an image of 300-600 micrometers rounded smooth metformin particles produced in the experiment. As shown, the surface of the particles prepared according to the teaching of the present embodiments was substantially smooth.

The metformin particles were subjected to a flow rate test using a Hall flow meter having a 5 millimeter diameter nozzle. The nozzle was tilted at an angle of 30°. For the powder containing the raw metformin particles, no flow was observed (zero flow). For the powder containing the rounded metformin particles, a flow rate of 10.2 gr/(cm²×s), was measured, demonstrating a vast improvement of flowability in the rounded metformin particles, compared to the raw particles.

### Example 5

### Production of Rounded Oxcarbazepine Particles

Rounded oxcarbazepine particles were prepared using an experimental system similar to the system shown in FIG. 8 and described in Examples 1 and 2 hereinabove.

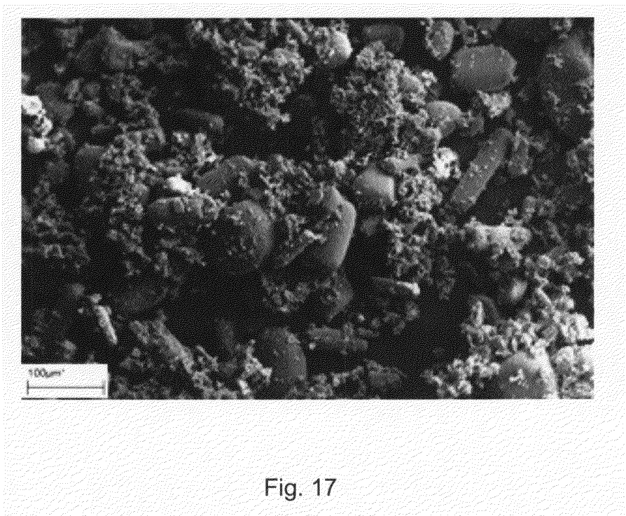

A 1.5 liter erlenmeyer flask was fastened to the rim of the liquid receiving container. 2.5 liter of acetone was poured into the erlenmeyer flask, and the volume between the erlenmeyer flask and the inner walls of the liquid receiving container was filled with water. 600 gr. of raw oxcarbazepine particles were added to the liquid in the erlenmeyer flask to form slurry. FIG. 16 is an image of the raw oxcarbazepine particles.

The ultrasonic transducers were activated while stirring the slurry. It was found by the inventors of the present invention that frequencies from about 16 kHz to about 60 kHz, and power densities from about 0.005 to about 0.05 kW/liter are suitable. Yet, higher efficiency was achieved at a frequency of about 25 kHz and a power density of about 0.01 kW/liter. During the process, a constant temperature of about 30° C. was maintained in liquid receiving container.

Following about 3.5 hours of treatment, the slurry was filtered under vacuum and the powder was dried at room temperature. A powder consisting of rounded smooth oxcarbazepine particles was formed.

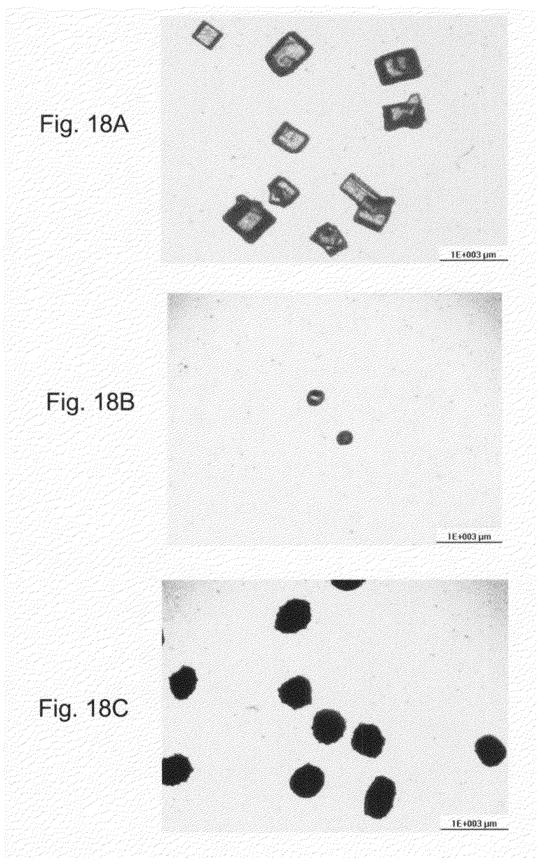

FIG. 17 is an image of rounded smooth oxcarbazepine particles produced in the experiment. Micronized particle population formed during the rounding process is also seen in the image.

### Example 6

### Sphericity and Shape Factor

The sphericity and shape factor of several samples of particles produced in various exemplary embodiments of the invention were measured.

The samples were imaged through a transmission optical microscope (magnification: ×12.5).

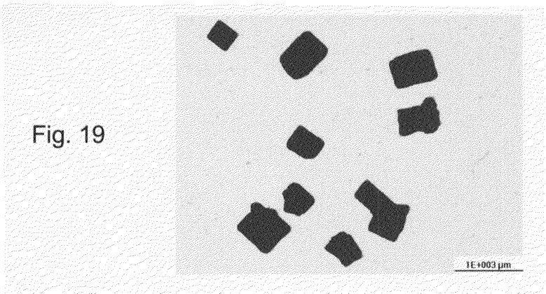

FIGS. 18A-C show the images of raw sucrose particles (FIG. 18A), 300-600 micrometer sucrose particles prepared as descried in Example 1 above (FIG. 18B), and 355-500 micrometer prior art Suglets® particles, purchased from NP Pharm, France (FIG. 18A).

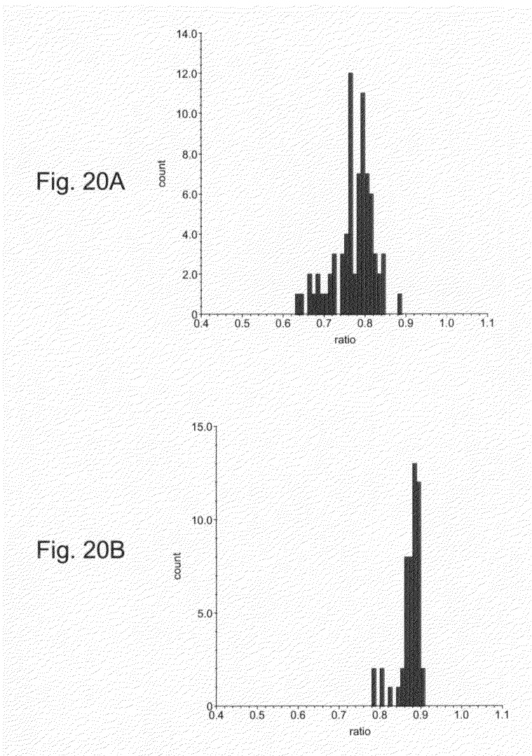

The images were analyzed by image processing software (Buehler Omnimet®). Each image processing comprised the following steps. In a first step the particles were delineated for 1 cycle; in a second step, dark areas were defined by thresholding; in a third step, the dark areas were filled; in a fourth step, particles smaller than 20×20 pixels were eliminated; in a fifth step border particles were eliminated; and in a sixth step the delineation of the non-eliminated particles were processed by the octagonal kernel of the software for 3 cycles. FIG. 19 is an image showing the raw sucrose particles after image processing.

Following image processing the area, A, and perimeter, P, of each particle was measured from the images. The sphericity of each particle was defined as 4πA/P².

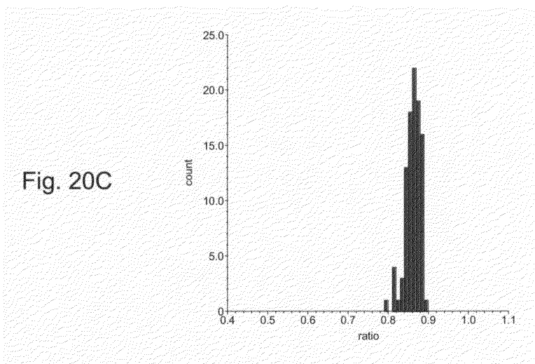

FIGS. 20A-C are histograms describing the sphericity as defined above for the samples shown in FIGS. 18A-C, respectively. The results of the three samples are summarized in Table 1, below.

TABLE 1

| Sample | No. of particles | sphericity |
|---|---|---|
| raw sucrose | 75 | 0.773 ± 0.05 |
| rounded 300-600 μm sucrose | 51 | 0.875 ± 0.027 |
| 355-500 μM Suglets ® (prior art) | 98 | 0.862 ± 0.019 |

As shown in Table 1, the present embodiments successfully produce round particles whose shape is sphere-like.

The sphericity of the particles was also measured using a DSA-10 image analyzer (Ankersmid Ltd., Israel). The analysis was performed during constant flow of slurry containing the particles. Images of about 1000 particles were analyzed by the image analyzer to determine the sphericity of the particles. In these measurements the sphericity was also as defined 4πA/P². To distinguish between the sphericity values obtained as determined using the image processing software and the sphericity values obtained using the image analyzer, the latter are referred to below as “shape factor”.

Beside sphericity, the DSA-10 image analyzer was used to characterize the shape of the particles by their aspect ratio, defined as the ratio between the minimal Feret and the maximal Feret of the particles.

Particles prepared in accordance with the teachings of the present embodiments shown improved shape characteristics in all categories. On the average, the particles prepared according to the teaching of the present embodiments shown a 5-20% enhancement of the shape factor and aspect ratio and a 10-35% enhancement of the sphericity, compared to the raw particles, demonstrating, again, the spherical nature of the particles of the embodiments.

Table 2 below summarizes the shape factor, aspect ratio and sphericity of several samples prepared in various exemplary embodiments of the invention.

TABLE 2

| | | No. of | shape | aspect | |
|---|---|---|---|---|---|
| Material | Sample | particles | factor | ratio | sphericity |
| raw dilitiazem | (i) | 110 | 0.7032 | 0.5416 | 0.5278 |
| hydrochloride | (ii) | 197 | 0.7056 | 0.5442 | 0.4773 |
| | (iii) | 226 | 0.6847 | 0.5293 | 0.4954 |
| rounded dilitiazem | (i) | 80 | 0.8211 | 0.6986 | 0.6601 |
| hydrochloride | (ii) | 33 | 0.8368 | 0.7053 | 0.6940 |
| above 45 μm | (iii) | 84 | 0.8229 | 0.7015 | 0.6572 |
| rounded dilitiazem | (i) | 164 | 0.8426 | 0.6915 | 0.5106 |
| hydrochloride | (ii) | 61 | 0.8168 | 0.6935 | 0.5220 |
| 20-45 μm | (iii) | 90 | 0.8467 | 0.6947 | 0.5139 |
| rounded dilitiazem | (i) | 73 | 0.8383 | 0.6828 | 0.5315 |
| hydrochloride | (ii) | 92 | 0.8419 | 0.6923 | 0.6004 |
| mix 20-45 μm + | (iii) | 164 | 0.8279 | 0.6731 | 0.5942 |
| 45 μm | | | | | |
| raw metformin | (i) | 40 | 0.8244 | 0.6900 | 0.6146 |
| | (ii) | 107 | 0.8214 | 0.6434 | 0.5675 |
| | (iii) | 85 | 0.8404 | 0.6420 | 0.5720 |
| rounded metformin | (i) | 54 | 0.8719 | 0.6791 | 0.7634 |
| | (ii) | 63 | 0.8739 | 0.6740 | 0.7676 |
| | (iii) | 122 | 0.8843 | 0.6904 | 0.7344 |
| raw oxcarbazepine | (i) | 141 | 0.7789 | 0.6102 | 0.4997 |
| | (ii) | 59 | 0.7908 | 0.6062 | 0.4760 |
| | (iii) | 177 | 0.7886 | 0.6221 | 0.4144 |
| rounded oxcarbazepine | (i) | 69 | 0.8138 | 0.5875 | 0.6103 |
| | (ii) | 102 | 0.7996 | 0.5887 | 0.5707 |
| | (iii) | 78 | 0.8174 | 0.5829 | 0.4969 |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A particle made of substance, the particle having a rounded shape and characterized by a substantially smooth surface.

2. A composition comprising a plurality of particles having a rounded shape and characterized by a substantially smooth surface.

3. A formulation, comprising a pharmaceutical composition and a particle made of substance, said particle having a rounded shape and characterized by a substantially smooth surface.

4. The particle of claim 1, wherein the substance comprises a pharmaceutically active ingredient.

5. The particle of claim 4, wherein said pharmaceutically active ingredient comprises diltiazem hydrochloride.

6. The particle of claim 4, wherein said pharmaceutically active ingredient comprises metformin.

7. The particle of claim 4, wherein said pharmaceutically active ingredient comprises oxcarbazepine.

8. The formulation of claim 3, wherein said particle is coated by said pharmaceutical composition.

9. The formulation of claim 3, wherein said particle is mixed with said pharmaceutical composition.

10. (canceled)

11. (canceled)

12. The particle of claim 1, wherein the substance comprises a pharmaceutically acceptable carrier.

13. The particle of claim 1, wherein the substance comprises a disaccharide.

14. The particle of claim 1, wherein said disaccharide is sucrose.

15. The particle of claim 1, wherein the substance comprises a monosaccharide.

16. The particle of claim 15, wherein said disaccharide is fructose.

17. The particle of claim 1, wherein the substance comprises at least one of a food substance, a nutritional substance and a nutraceutical substance.

18. (canceled)

19. (canceled)

20. The particle claim 1, wherein said substance comprises at least one of a cocoa and an instant coffee component.

21. (canceled)

22. The particle of claim 1, wherein said substance comprises at least one of a vitamin and a mineral.

23. (canceled)

24. (canceled)

25. (canceled)

26. The particle or of claim 1, wherein the particle consists of a single substance.

27. The particle of claim 26, characterized by a non-layered filled structure.

28. (canceled)

29. The particle of claim 1, wherein said rounded shape is characterized by a sphericity of at least 80%.

30. The particle of claim 1, wherein the particle has a specific surface area being lower than 0.1 m² per gram per 400 micrometer diameter.

31. The particle of claim 1, wherein said substantially smooth surface is characterized by a roughness being lower than 1% of the diameter of the particle.

32. The particle of claim 1, wherein a ratio between an optical surface area of the particle and a BET surface area of the particle is larger than 0.3.

33. (canceled)

34. The composition of claim 2, being a dry powder composition characterized by an angle of repose which is lower than 45 degrees.

35. The composition of claim 2, being a dry powder composition capable of maintaining a flow rate of at least 6 gram per square centimeter per second, through a 5 millimeters nozzle tilted at an angle of 30°.
